(19) United States
(12) Patent Application Publication
(10) Pub. No.: US 2021/0151796 A1
(43) Pub. Date: May 20, 2021

(54) Electrolytic Solution for Lithium Secondary Batteries and Lithium Secondary Battery Including the Same

(71) Applicants: Hyundai Motor Company, Seoul (KR); Kia Motors Corporation, Seoul (KR); The Industry & Academic Cooperation in Chungnam National University (IAC), Daejeon (KR)

(72) Inventors: Yoon Sung Lee, Suwon-si (KR); Yeol Mae Yeo, Hwaseong-si (KR); Seung Min Oh, Incheon (KR); Ik Kyu Kim, Gwangmyeong-si (KR); Ji Eun Lee, Hwaseong-si (KR); Nam Hyeong Kim, Gimcheon-si (KR); Dong Jun Kim, Seongnam-si (KR); Seung Wan Song, Sejong-si (KR); Gyeong Jun Chung, Daejeon (KR)

(21) Appl. No.: 16/882,842

(22) Filed: May 26, 2020

(30) Foreign Application Priority Data
Nov. 20, 2019 (KR) 10-2019-0149812

(51) Int. Cl. H01M 10/0567; H01M 10/052; H01M 4/38; H01M 4/587; H01M 4/505; H01M 4/525; C07C 69/96

(52) U.S. Cl. CPC H01M 10/0567; H01M 10/052; H01M 4/386; H01M 2300/0025; H01M 4/505; H01M 4/525; C07C 69/96; H01M 4/587

(57) ABSTRACT

An electrolytic solution is disclosed. The electrolytic solution can be used, for example, in a lithium secondary battery. The electrolytic solution includes lithium salt, a solvent, and a functional additive, wherein the functional additive comprises a high-voltage additive, and wherein the high-voltage additive comprises 1-fluoroethyl methyl carbonate (FEMC), expressed by the following formula:

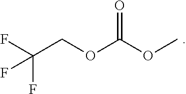

### CROSS-REFERENCE TO RELATED APPLICATIONS

This application claims priority to Korean Patent Application No. 10-2019-0149812, filed in the Korean Intellectual Property Office on Nov. 20, 2019, which application is hereby incorporated herein by reference.

### TECHNICAL FIELD

The present invention relates to an electrolytic solution for lithium secondary batteries and a lithium secondary battery including the same.

### BACKGROUND

A lithium secondary battery is an energy storage including a positive electrode configured to provide lithium during charging, a negative electrode configured to receive lithium during charging, an electrolyte serving as a lithium ion transfer medium, and a separator configured to separate the positive electrode and the negative electrode from each other. The lithium secondary battery generates and stores electric energy through a change in chemical potential when lithium ions are intercalated/deintercalated at the positive electrode and the negative electrode.

The lithium secondary battery is mainly used in portable electronic devices. In recent years, however, the lithium secondary battery has been used as an energy storage means of an electric vehicle (EV) and a hybrid electric vehicle (HEV) as the electric vehicle and the hybrid electric vehicle are commercialized.

Research to increase the energy density of the lithium secondary battery in order to increase the travel distance of the electric vehicle has been conducted. The energy density of the lithium secondary battery may be increased by increasing the capacity of the positive electrode.

The capacity of the positive electrode may be increased by using a Ni-rich method, which is a method of increasing the content of Ni of a Ni—Co—Mn oxide forming a positive electrode active material, or by increasing the positive electrode charging voltage.

However, the Ni-rich Ni—Co—Mn oxide has an unstable crystalline structure while exhibiting high interfacial reactivity, whereby degradation during cycles is accelerated and thus it is difficult to secure long-term performance of the lithium secondary battery.

The matters disclosed in this section are merely for enhancement of understanding of the general background of the invention and should not be taken as an acknowledgment or any form of suggestion that the matters form the related art already known to a person skilled in the art.

### SUMMARY

The present invention relates to an electrolytic solution for lithium secondary batteries and a lithium secondary battery including the same. Embodiments of the present invention address the above problems. Particular embodiments of the present invention provide an electrolytic solution for lithium secondary batteries capable of improving lifespan and output characteristics of lithium secondary batteries and a lithium secondary battery including the same.

In accordance with an embodiment of the present invention, the above and other objects can be accomplished by the provision of an electrolytic solution for lithium secondary batteries, the electrolytic solution including lithium salt, a solvent, and a functional additive, wherein the functional additive includes a high-voltage additive, 1-fluoroethyl methyl carbonate (FEMC), expressed by [Formula 1] below.

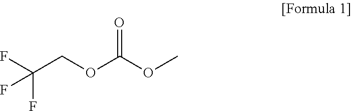

The high-voltage additive may be added so as to account for 1 to 3 wt % based on the weight of the electrolytic solution.

The functional additive may further include a negative electrode film additive, such as vinylene carbonate (VC).

The negative electrode film additive may be added so as to account for 0.5 to 3.0 wt % based on the weight of the electrolytic solution.

The lithium salt may be any one or a mixture of two or more selected from the group consisting of LiPF₆, LiBF₄, LiClO₄, LiCl, LiBr, LiI, LiB₁₀Cl₁₀, LiCF₃SO₃, LiCF₃CO₂, LiAsF₆, LiSbF₆, LiAlCl₄, CH₃SO₃Li, CF₃SO₃Li, LiN(SO₂C₂F₅)₂, Li(CF₃SO₂)₂N, LiC₄F₉SO₃, LiB(C₆H₅)₄, Li(SO₂F)₂N(LiFSI), and (CF₃SO₂)₂NLi.

The solvent may be any one or a mixture of two or more selected from the group consisting of a carbonate-based solvent, an ester-based solvent, an ether-based solvent, and a ketone-based solvent.

In accordance with another embodiment of the present invention, there is provided a lithium secondary battery including an electrolytic solution. The lithium secondary battery may further include a positive electrode including a positive electrode active material consisting of Ni, Co, and Mn, a negative electrode including one or more negative electrode active materials selected from among carbon (C)-based and silicon (Si)-based negative electrode active materials, and a separator interposed between the positive electrode and the negative electrode.

The content of Ni in the positive electrode may be 60 wt % or more.

### BRIEF DESCRIPTION OF THE DRAWINGS

The above and other objects, features and other advantages of the present invention will be more clearly understood from the following detailed description taken in conjunction with the accompanying drawings, in which:

FIGS. 1 to 3 are graphs showing the results of charge and discharge of Examples and Comparative Examples;

FIG. 4 is a photograph showing the surfaces of positive electrodes after charge and discharge of Examples and Comparative Examples; and

FIG. 5 is a simple diagram representing a lithium secondary battery according to an embodiment of the present invention.

### DETAILED DESCRIPTION OF ILLUSTRATIVE EMBODIMENTS

Hereinafter, exemplary embodiments of the present invention will be described in detail with reference to the accompanying drawings. However, the present invention is not limited to the embodiments disclosed below and may be implemented in various different forms, and the embodiments herein are provided to make the disclosure of the present invention complete and to fully convey the scope of the invention to those skilled in the art.

An electrolytic solution for lithium secondary batteries according to an embodiment of the present invention is a material that forms an electrolyte applied to a lithium secondary battery, and includes lithium salt, a solvent, and a functional additive.

The lithium salt may be any one or a mixture of two or more selected from the group consisting of LiPF₆, LiBF₄, LiClO₄, LiCl, LiBr, LiI, LiB₁₀Cl₁₀, LiCF₃SO₃, LiCF₃CO₂, LiAsF₆, LiSbF₆, LiAlCl₄, CH₃SO₃Li, CF₃SO₃Li, LiN(SO₂C₂F₅)₂, Li(CF₃SO₂)₂N, LiC₄F₉SO₃, LiB(C₆H₅)₄, Li(SO₂F)₂N(LiFSI), and (CF₃SO₂)₂NLi.

The lithium salt may be present in the electrolytic solution such that the total amount of the lithium salt has a concentration of 0.1 to 1.2 moles.

Any one or a mixture of two or more selected from the group consisting of a carbonate-based solvent, an ester-based solvent, an ether-based solvent, and a ketone-based solvent may be used as the solvent.

Dimethyl carbonate (DMC), diethyl carbonate (DEC), dipropyl carbonate (DPC), methyl propyl carbonate (MPC), ethyl propyl carbonate (EPC), ethyl methyl carbonate (EMC), ethylene carbonate (EC), propylene carbonate (PC), butylene carbonate (BC), fluoroethylene carbonate (FEC), or vinylene carbonate (VC) may be used as the carbonate-based solvent. γ-butyrolactone (GBL), n-methyl acetate, n-ethyl acetate, or n-propyl acetate may be used as the ester-based solvent. Dibutyl ether may be used as the ether-based solvent. However, the present invention is not limited thereto.

In addition, the solvent may further include an aromatic hydrocarbon-based organic solvent. Concrete examples of the aromatic hydrocarbon-based organic solvent may include benzene, fluorobenzene, bromobenzene, chlorobenzene, cyclohexylbenzene, isopropylbenzene, n-butylbenzene, octylbenzene, toluene, xylene, and mesitylene, which may be used either alone or as a mixture of two or more.

Meanwhile, a high-voltage additive expressed by [Formula 1] below, such as 1-fluoroethyl methyl carbonate (FEMC), may be used as the functional additive added to the electrolytic solution according to an embodiment of the present invention.

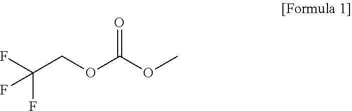

The FEMC serves to improve oxidative stability of the electrolytic solution and to stabilize the interface between the electrolytic solution and a positive electrode, and may be added so as to account for 1 to 3 wt % based on the weight of the electrolytic solution.

In the case in which the content of the high-voltage additive is less than 1 wt %, it is difficult to sufficiently form a surface protective layer, whereby the expected effect is deficient. In the case in which the content of the high-voltage additive is greater than 3 wt %, the surface protective layer is excessively formed, whereby cell resistance is increased and thus battery output is reduced.

Meanwhile, a negative electrode film additive serving to form a film on a negative electrode may be further added as the functional additive. For example, vinylene carbonate (VC) may be used as the negative electrode film additive.

The negative electrode film additive may be added so as to account for preferably 0.5 to 3.0 wt %, more preferably 1.5 to 2.5 wt %, based on the weight of the electrolytic solution.

In the case in which the content of the negative electrode film additive is less than 0.5 wt %, the long-term lifespan characteristics of the cell are deteriorated. In the case in which the content of the negative electrode film additive is greater than 3.0 wt %, the surface protective layer is excessively formed, whereby cell resistance is increased and thus battery output is reduced.

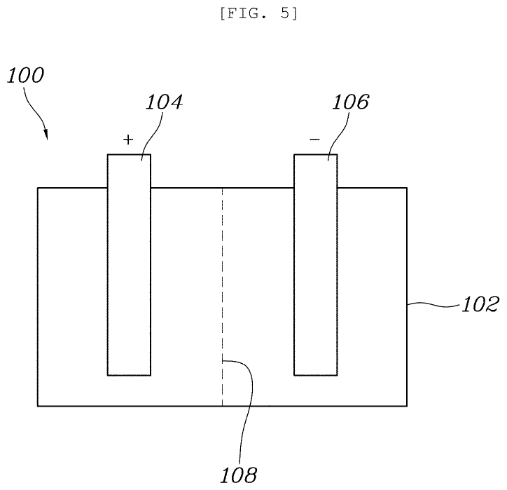

As illustrated in FIG. 5, a lithium secondary battery 100 according to an embodiment of the present invention includes a battery housing 102, a positive electrode 104 having a portion within the battery housing 102, a negative electrode 106 having a portion within the battery housing 102, a separator 108 interposed between the positive electrode 104 and the negative electrode 106, and the electrolytic solution as described herein within the battery housing 102.

The positive electrode 104 includes an NCM-based positive electrode active material consisting of Ni, Co, and Mn. Particularly, in this embodiment, the positive electrode active material included in the positive electrode 104 may include only an NCM-based positive electrode active material containing 60 wt % or more of Ni.

The negative electrode 106 includes one or more negative electrode active materials selected from among carbon (C)-based and silicon (Si)-based negative electrode active materials.

At least one selected from the group consisting of artificial graphite, natural graphite, graphitized carbon fibers, graphitized mesocarbon mircobeads, fullerene, and amorphous carbon may be used as the carbon (C)-based negative electrode active materials.

The silicon (Si)-based negative electrode active materials include a silicon oxide, silicon particles, and silicon alloy particles.

Meanwhile, each of the positive electrode 104 and the negative electrode 106 is manufactured by mixing a conductive agent, a binder, and a solvent with the active material thereof to manufacture an electrode slurry and directly coating and drying the electrode slurry on a current collector. Aluminum (Al) may be used as the current collector. However, embodiments of the present invention are not limited thereto. An electrode manufacturing method is well known in the art to which the present invention pertains, and therefore a detailed description will be omitted from this specification.

The binder serves to properly attach active material particles to each other or to properly attach the active material particles to the current collector, and examples of the binder may include, but are not limited to, polyvinyl alcohol, carboxymethyl cellulose, hydroxypropyl cellulose, diacetyl cellulose, polyvinyl chloride, carboxylated polyvinyl chloride, polyvinyl fluoride, a polymer including an ethylene oxide, polyvinylpyrrolidone, polyurethane, polytetrafluoroethylene, polyvinylidene fluoride, polyethylene, polypropylene, styrene-butadiene rubber, acrylated styrene-butadiene rubber, an epoxy resin, and nylon.

In addition, the conductive agent is used to provide conductivity to an electrode, and any conductive agent may be used as long as the conductive agent does not cause a chemical change in a battery and is made of an electrically conductive material. For example, natural graphite, artificial graphite, carbon black, acetylene black, Ketjen black, carbon fiber, or metal powder or metal fiber, such as copper, nickel, aluminum, or silver may be used as the conductive agent. In addition, conductive materials, such as polyphenylene derivatives, may be used either alone or as a mixture of two or more.

The separator 108 prevents short circuit between the positive electrode 104 and the negative electrode 106, and provides a movement path for lithium ions. A polyolefin-based polymer film, such as polypropylene, polyethylene, polyethylene/polypropylene, polyethylene/polypropylene/polyethylene, or polypropylene/polyethylene/polypropylene, a multilayered film, a microporous film, woven fabric, or non-woven fabric may be used as the separator. In addition, a film obtained by coating a resin exhibiting high stability on a porous polyolefin film may be used.

Hereinafter, the present invention will be described based on Examples and Comparative Examples.

<Experiment 1> Experiment on Characteristics by Voltage at Room Temperature (25° C.) Based on Kind of Functional Additive (Half Cell)

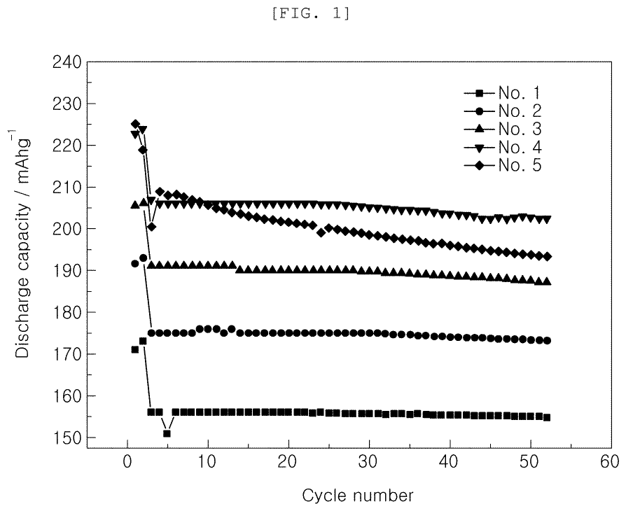

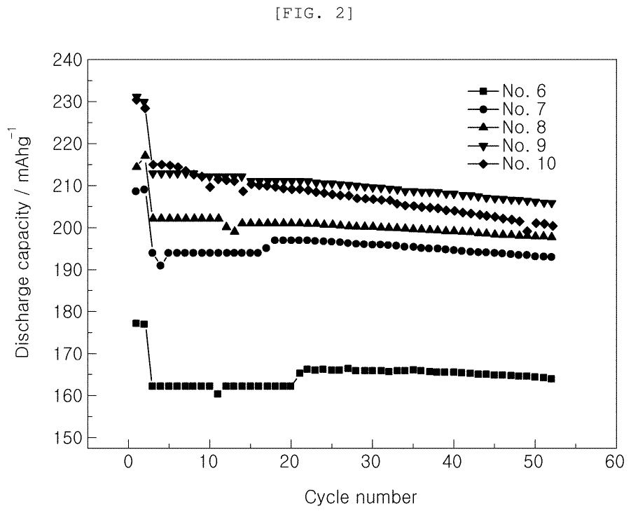

In order to investigate characteristics by voltage based on the kind of the functional additive added to the electrolytic solution, initial capacities and capacity retentions at room temperature (25° C.) were measured while changing the kind of the functional additive and voltage, as shown in Table 1 below. The results are shown in Table 1 and FIGS. 1 and 2.

In order to manufacture the electrolytic solution, 0.5M LiPF₆ and 0.5M LiFSI were used as the lithium salt, and a mixture of ethylene carbonate (EC):ethyl methyl carbonate (EMC):diethyl carbonate (DEC) in a ratio of 25:45:30 was used as the solvent.

NCM622 was used as the positive electrode, and Li metal was used as the negative electrode.

TABLE 1

| | | | | | Initial capacity | Capacity retention |
|---|---|---|---|---|---|---|
| | | Additive | | | @1 C 1st cyc | @1 C 50 cyc |
| Classification | | VC | FEMC | Voltage | (mAh/g) | (%) |
| No. 1 | Comparative | 2 | — | 4.2 | 156 | 99.2 |
| | Example | | | | | |
| No. 2 | Comparative | 2 | — | 4.3 | 175 | 98.9 |
| | Example | | | | | |
| No. 3 | Comparative | 2 | — | 4.4 | 191 | 98 |
| | Example | | | | | |
| No. 4 | Comparative | 2 | — | 4.5 | 207 | 97.7 |
| | Example | | | | | |
| No. 5 | Comparative | 2 | — | 4.6 | 200 | 96.5 |
| | Example | | | | | |
| No. 6 | Example | — | 2 | 4.2 | 162 | 101 |
| No. 7 | Example | — | 2 | 4.3 | 194 | 99.5 |
| No. 8 | Example | — | 2 | 4.4 | 202 | 97.9 |
| No. 9 | Example | — | 2 | 4.5 | 212 | 97.1 |
| No. 10 | Example | — | 2 | 4.6 | 215 | 93.2 |

It can be seen from Table 1 and FIGS. 1 and 2 that, in the case in which FEMC according to Examples was used as the functional additive, initial capacities increased more than in the case in which only VC, which is a conventional general functional additive, was used, under the same voltage condition.

In addition, it can be seen that, in the case in which the same functional additive was used, cell capacities increased through initial revelation of high capacities as voltage was increased and high capacity retentions were maintained at voltages ranging from 4.2 V to 4.5 V.

<Experiment 2> Experiment on Charge and Discharge Characteristics at High Temperature (45° C.) Based on Kind and Content of Functional Additive (Half Cell)

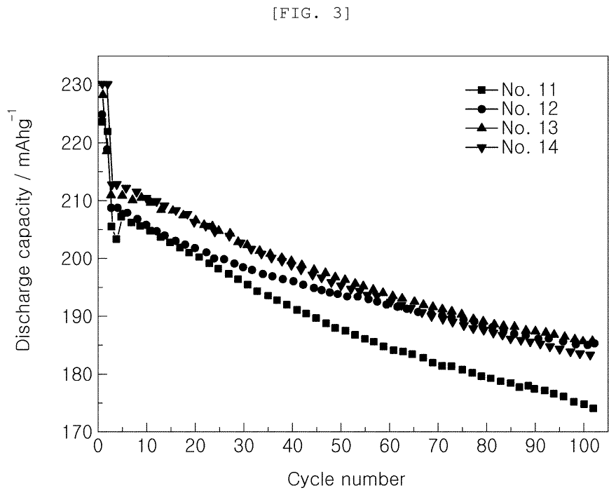

In order to investigate charge and discharge characteristics based on the kind and content of the functional additive added to the electrolytic solution, initial capacities and capacity retentions at high temperature (45° C.) were measured while changing the kind and content of the functional additive, as shown in Table 2 below. The results are shown in Table 2 and FIG. 3.

TABLE 2

| | | | | Initial capacity | Capacity retention |
|---|---|---|---|---|---|
| | | Additive | | @1 C 1st cyc | @1 C 50 cyc |
| Classification | | VC | FEMC | (mAh/g) | (%) |
| No. 11 | Comparative | 2 | — | 205 | 84.5 |
| | Example | | | | |
| No. 12 | Example | 2 | 1 | 208 | 88.7 |
| No. 13 | Example | 2 | 2 | 210 | 88.9 |
| No. 14 | Example | 2 | 3 | 212 | 86.2 |

It can be seen from Table 2 and FIG. 3 that, in the case in which VC, which is a conventional general functional additive, was used and FEMC according to Examples was used, while the content of the functional additive was changed, initial capacities increased as the content of FEMC was increased.

In addition, it can be seen that, in the case in which FEMC according to Examples was used as the functional additive, higher capacity retentions were maintained than in the case in which only VC, which is a conventional general functional additive, was used.

<Experiment 3> Experiment on Analysis of Surface of Positive Electrode Before and after Charge and Discharge by Kind of Functional Additive

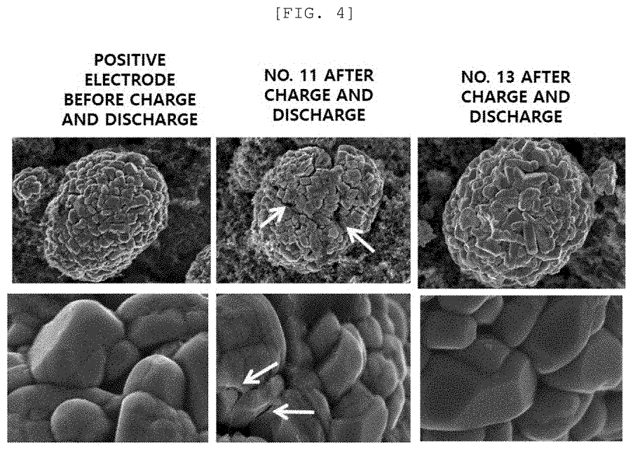

Surfaces before and after charge and discharge experiments at high temperature (45° C.) in the case in which the electrolytic solutions according to No. 11 and No. 13 were used were observed, and the results are shown in FIG. 4.

It can be seen from FIG. 4 that, in the case of No. 11, in which only VC, which is a conventional general functional additive, was used as the functional additive, cracks were formed in the positive electrode.

However, it can be seen that, in the case of No. 13, in which FEMC, which is a functional additive according to the present invention, was used as the functional additive, no cracks were formed in the positive electrode.

As is apparent from the above description, according to embodiments of the present invention, an electrolytic solution including a high-voltage additive is used, whereby the long-term lifespan characteristics of a lithium secondary battery are improved.

In addition, in the case in which the electrolytic solution including the high-voltage additive is used, cell resistance of the lithium secondary battery is reduced, whereby the output characteristics of the lithium secondary battery are improved.

Although the preferred embodiments of the present invention have been described above with reference to the accompanying drawings, those skilled in the art will appreciate that the present invention can be implemented in various other embodiments without changing the technical ideas or features thereof.

What is claimed is:

1. An electrolytic solution for lithium secondary batteries, the electrolytic solution comprising:
lithium salt;
a solvent; and
a functional additive that comprises a high-voltage additive, wherein the high-voltage additive comprises 1-fluoroethyl methyl carbonate (FEMC), expressed by [Formula 1]

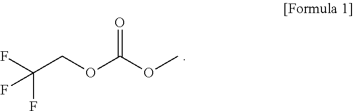

2. The electrolytic solution according to claim 1, wherein the high-voltage additive accounts for 1 to 3 wt % based on a weight of the electrolytic solution.

3. The electrolytic solution according to claim 1, wherein the functional additive further comprises a negative electrode film additive, and wherein the negative electrode film additive comprises vinylene carbonate (VC).

4. The electrolytic solution according to claim 3, wherein the negative electrode film additive accounts for 0.5 to 3.0 wt % based on a weight of the electrolytic solution.

5. The electrolytic solution according to claim 1, wherein the lithium salt is any one or a mixture of two or more materials selected from the group consisting of LiPF₆, LiBF₄, LiClO₄, LiCl, LiBr, Li, LiB₁₀Cl₁₀, LiCF₃SO₃, LiCF₃CO₂, LiAsF₆, LiSbF₆, LiAlCl₄, CH₃SO₃Li, CF₃SO₃Li, LiN(SO₂C₂F₅)₂, Li(CF₃SO₂)₂N, LiC₄F₉SO₃, LiB(C₆H₅)₄, Li(SO₂F)₂N(LiFSI), and (CF₃SO₂)₂NLi.

6. The electrolytic solution according to claim 1, wherein the solvent is any one or a mixture of two or more materials selected from the group consisting of a carbonate-based solvent, an ester-based solvent, an ether-based solvent, and a ketone-based solvent.

7. A lithium secondary battery comprising an electrolytic solution, the electrolytic solution comprising lithium salt, a solvent, and a functional additive, wherein the functional additive comprises a high-voltage additive, and wherein the high-voltage additive comprises 1-fluoroethyl methyl carbonate (FEMC) expressed by

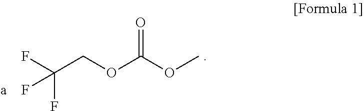

8. The lithium secondary battery according to claim 7, further comprising:
a positive electrode comprising a positive electrode active material selected from the group consisting of Ni, Co, and Mn;
a negative electrode comprising one or more negative electrode active materials selected from among carbon (C)-based or silicon (Si)-based negative electrode active materials; and
a separator interposed between the positive electrode and the negative electrode.

9. The lithium secondary battery according to claim 8, wherein a content of Ni in the positive electrode is 60 wt % or more.

10. A lithium secondary battery comprising:
a battery housing;
a positive electrode having a portion within the battery housing;
a negative electrode having a portion within the battery housing;
a separator interposed between the positive electrode and the negative electrode; and
an electrolytic solution within the battery housing, wherein the electrolytic solution comprises lithium salt, a solvent, and a functional additive, the functional additive comprising a high-voltage additive expressed by [Formula 1]

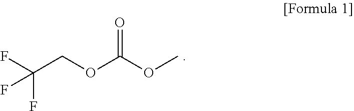

11. The lithium secondary battery according to claim 10, wherein the positive electrode includes an NCM-based positive electrode active material comprising Ni, Co, and Mn.

12. The lithium secondary battery according to claim 11, wherein the NCM-based positive electrode active material comprises 60 wt % or more of Ni.

13. The lithium secondary battery according to claim 10, wherein the negative electrode comprises one or more negative electrode active materials, wherein the negative electrode active materials are carbon-based negative electrode active materials or silicon-based negative electrode active materials.

14. The lithium secondary battery according to claim 13, wherein the carbon-based negative electrode active materials include at least one of artificial graphite, natural graphite, graphitized carbon fibers, graphitized mesocarbon mircobeads, fullerene, or amorphous carbon, and wherein the silicon-based negative electrode active materials include a silicon oxide, silicon particles, or silicon alloy particles.

15. The lithium secondary battery according to claim 10, wherein the separator comprises a polyolefin-based polymer film, a multilayered film, a microporous film, woven fabric, or non-woven fabric.

16. The lithium secondary battery according to claim 10, wherein the high-voltage additive comprises 1-fluoroethyl methyl carbonate (FEMC).

17. The lithium secondary battery according to claim 10, wherein the functional additive comprises a negative electrode film additive, the high-voltage additive accounts for 1 to 3 wt % based on a weight of the electrolytic solution, and the negative electrode film additive accounts for 0.5 to 3.0 wt % based on a weight of the electrolytic solution.

18. The lithium secondary battery according to claim 17, wherein the negative electrode film additive comprises vinylene carbonate (VC).

19. The lithium secondary battery according to claim 10, wherein the lithium salt is any one or a mixture of two or more materials selected from the group consisting of LiPF₆, LiBF₄, LiClO₄, LiCl, LiBr, Li, LiB₁₀Cl₁₀, LiCF₃SO₃, LiCF₃CO₂, LiAsF₆, LiSbF₆, LiAlCl₄, CH₃SO₃Li, CF₃SO₃Li, LiN(SO₂C₂F₅)₂, Li(CF₃SO₂)₂N, LiC₄F₉SO₃, LiB(C₆H₅)₄, Li(SO₂F) 2N (LiFSI), and (CF₃SO₂)₂NLi.

20. The lithium secondary battery according to claim 10, wherein the solvent is any one or a mixture of two or more materials selected from the group consisting of a carbonate-based solvent, an ester-based solvent, an ether-based solvent, and a ketone-based solvent.
